(19) United States
(12) Patent Application Publication
(10) Pub. No.: US 2017/0246261 A1
(43) Pub. Date: Aug. 31, 2017

(54) ANTIVIRAL TREATMENT WITH LOW IMMUNOGENICITY

(71) Applicant: Agenovir Corporation, South San Francisco, CA (US)

(72) Inventors: Ed Mocarski, South San Francisco, CA (US); Stephen R. Quake, Stanford, CA (US); Xin Cindy Xiong, San Mateo, CA (US)

(21) Appl. No.: 15/441,677

(22) Filed: Feb. 24, 2017

Related U.S. Application Data
(60) Provisional application No. 62/299,829, filed on Feb. 25, 2016.

(51) Int. Cl. A61K 38/46; A61K 45/06

(52) U.S. Cl. CPC A61K 38/465; A61K 45/06; A61K 48/00

(57) ABSTRACT

Compositions and methods are disclosed for reducing toxicity and immunogenicity of nucleases, especially when in use for cutting viral nucleic acids in host cells. Different nucleases that cut the same target are delivered at different times to avoid an immune response that interferes with a therapeutic effect of the nucleases.

### CROSS-REFERENCE TO RELATED APPLICATION

This application claims benefit of U.S. Provisional Application Ser. No. 62/299,829, filed Feb. 25, 2016, incorporated by reference.

### TECHNICAL FIELD

The invention relates to reducing immunogenicity of gene editing tools in antiviral treatments and other applications.

### BACKGROUND

It has been recently proposed that certain nucleases have utility as gene-editing therapeutics. In particular, the CRISPR/Cas system has been widely reported as a tool for targeted editing of genomic DNA. However, therapeutic nucleases may prove challenging because the nucleases proposed for a therapeutic role are derived from bacteria and may be recognized as “foreign” molecules by the human immune system.

The use of a foreign molecule as a therapeutic may result in an anti-drug antibody response that reduces or eliminates the effectiveness of the therapeutic or, in extreme cases, causes an allergic response. In order for nuclease therapy to be effective, it must be administered in a manner that results in the avoidance of a significant immune response by the host.

### SUMMARY

The invention provides compositions and methods for administering a gene-editing nuclease with reduced intracellular toxicity mediated by protein-protein interactions. Specifically, the invention provides methods for introducing gene-editing nucleases in a manner that avoids an immune response that negates the therapeutic effect or that triggers a significant immune response to the therapeutic itself.

In a preferred embodiment, the invention comprises the administration of a plurality of nucleases that cleave the same target but that are not cross immunogenic. In other words, the nucleases have the same therapeutic effect but do not trigger cross-reacting antibodies. Ideally, the nucleases are administered serially in connection with a gene-editing cassette, such as the CRISPR/Cas9 cassette, so that they are targeted to the same region of the genome.

Pharmaceutical compositions of the invention can be programmed to target any genomic region as detailed below. A preferred target is viral nucleic acid. Either integrated or non-integrated virus can be targeted with the endpoint of editing viral nucleic acid in order to disrupt viral function and/or replication. Since the plurality of nucleases are different, they present different antigens to an immune system of the cells. Thus even if the immune system is primed by the first nuclease, the second nuclease is not in the immunological memory of the immune system. The invention provides for the successful delivery of nucleases to cells while avoiding an immune response that would diminish therapeutic effects of the nucleases.

Embodiments of the invention may further include reducing immunogenicity or toxicity of nuclease treatment by such measures as: modification of the nuclease, treating cells or tissue ex vivo and delivering a product to a patient, careful measurement of viral load, and measurement of treatment byproducts such as proteins and on-target and off-target cut nucleic acids. Such measures allow for the precise dosing of the gene-editing nuclease, which will minimize general toxicity or immunogenicity. Methods of the invention also contemplate co-administration of an immunosuppressant, such as prednisolone or others known in the art. In addition, other measures can be taken to ensure that there is no immune response to the endonuclease therapy, such as the modification of dosage schedule and amount to reduce anti-drug antibody production; the utilization of alternative routes of delivery (e.g., blood, gut, mucosal, oral, nasal, etc.); and using modified nucleases.

In certain aspects, the invention provides methods for delivering a therapeutic nuclease. Preferred methods include introducing a first nuclease that cuts a target site in a target nucleic acid and a second nuclease that cuts the same target site, wherein the first and second nucleases are not immunogenically cross-reactive. The first, second, and any subsequent nucleases may differ by being of different types, by modifications, or by being from different bacterial species. For example, two or more of a Cas-type nuclease, a transcription-activator like effector domain nuclease (TALEN), and a zinc-finger nuclease may be used. Additionally or alternatively, a nuclease and modified version of that nuclease may be used. Further, different Cas-type nucleases from different species may be used such as a combination of Cas6, Cas9, and Cpf1.

Therapeutic compositions of the invention are useful to target nucleic acid generally. More specifically, compositions of the invention are useful for therapeutic gene editing. One area of use is the editing of viral nucleic acid. Compositions of the invention are delivered to virally-infected cells and the nuclease portion of the composition cleaves viral nucleic acid in order to inactivate the virus and/or prevent it from replicating.

The nuclease may be provided as a protein, a ribonucleoprotein (RNP), mRNA, or by delivering DNA vectors such as plasmids or AAV vectors that encode the first nuclease and the second nuclease. The nucleic acid encoding the first or second nuclease may be introduced into the cell by different means selected from the group consisting of: clonal micelle, liposome, extracellular vesicle, nanoparticle, copolymer block, adeno-associated virus, virus-like particle, and adenovirus.

Where, for example, the nucleases are Cas-type nucleases, such as Cas9 and variants thereof, DNA vectors may each encode a guide RNA complementary to the nucleic acid target, wherein the first nuclease and the second nuclease each form a complex a transcript of the guide RNA to specifically cut the target site. The plurality of nucleases may, for example, each have at least 80% sequence identity to Cas9 but should not be identical.

In some embodiments, a modified nuclease not known to occur in nature is used. The modified nuclease may be smaller than a wild type counterpart. The modified nuclease may be modified by removal of nonfunctional structures of the wild type counterpart. The modified nuclease may have an altered charge and/or hydrophobicity from a wild type counterpart. The modified nuclease may be a fusion protein comprising a portion of a protein selected from the group consisting of: GFP, Fc, and IgG.

The method may further include assaying for viral load in the cells and determining an amount of each nuclease to be delivered based on the viral load. The method may include assaying for viral load in the cell before delivering the first gene-editing therapeutic dose and determining the first gene-editing therapeutic dose based on the viral load. Methods of the invention may include assaying for viral load in the cell before delivering a second (or subsequent) gene-editing therapeutic dose and determining the second gene-editing therapeutic dose based on the viral load. The nucleic acid encoding the first nuclease and the nucleic acid encoding the second (and any subsequent) nucleic acid may be introduced into the cell by different means selected from the group consisting of: clonal micelle, liposome, extracellular vesicle, nanoparticle, copolymer block, adeno-associated virus, virus-like particle, and adenovirus.

In certain aspects, methods of the invention include treating cells of a patient with a nuclease that preferentially cuts nucleic acid of a virus over patient nucleic acid (not including viral nucleic acid if the virus is integrated). Methods may include assaying for viral load in the patient before treatment and determining a first nuclease dose based on the viral load. In various embodiments, methods may include assaying for viral load in the patient after treatment and determining a second (or subsequent) nuclease dose based on the viral load after treatment, the viral load before treatment, and the first nuclease dose.

In various embodiments, methods may include assaying a patient sample after a first nuclease dose to determine an amount of foreign material introduced by the first nuclease dose.

The assaying step may include determining protein products present in the patient sample. The assay may include flow cytometry, immunoassay, or ELISA assay. The assaying step may include measuring a level of a protein in the patient sample, wherein the protein is known to be affected by the nuclease cut nucleic acid. In certain embodiments, the assay step can include measuring levels of cut viral nucleic acid and cut patient nucleic acid.

In certain embodiments of the invention, the nuclease comprises Cas9 complexed with a guide RNA complementary to a portion of the viral nucleic acid. The guide RNA may be at least 20 mer. In some methods of the invention, Cas9 is modified from the wild type. The modified Cas9 may be smaller than wild type Cas9. Nonfunctional structures may have been removed from the modified Cas9 and the functionality may have been determined experimentally. The Cas9 may have been modified through random mutagenesis. In certain embodiments, the modified Cas9 may be a fusion protein fused with another protein or portion thereof. In certain embodiments, the other protein may comprise, GFP, Fc, or IgG. The modified protein, including potential fusion proteins may have an altered charge and/or hydrophobicity relative to wild type Cas9.

In various embodiments, cells obtained from a patient may be treated ex vivo and introduced into the patient after treatment. Treating the cells may comprise introduction of the nuclease into the cells through: clonal micelle, liposome, extracellular vesicle, nanoparticle, copolymer block, adeno-associated virus, virus-like particle, and adenovirus. Treating the cells may include introduction of mRNA configured to synthesize the nuclease into the cells. In various embodiments, the virus may be an oncovirus and the patient may be diagnosed with: lymphoma, nasopharyngeal carcinoma, gastrointestinal carcinoma, lethal midline granuloma, cervical carcinoma, oropharyngeal carcinoma, anal carcinoma, or Merkel cell carcinoma.

Aspects of the invention include methods for treating viruses including treating cells of a patient with a first dose comprising a first nuclease that cuts nucleic acid of a virus and a second dose comprising a second nuclease that cuts nucleic acid of the virus where the first nuclease and the second nuclease differ by at least one amino acid residue. In certain embodiments, the first nuclease and the second nuclease may originate from different species.

### BRIEF DESCRIPTION OF THE DRAWINGS

FIG. 1 diagrams steps of certain methods of the invention.

FIG. 2 shows a composition that includes an EGFP marker fused after the Cas9 protein.

FIG. 3 shows gRNA targets along a reference genome.

FIG. 4 shows a system, including an ultrasound transducer, for removing target genetic material from a subject according to certain embodiments.

FIG. 5 shows a system, including an electroporation device, for removing target genetic material from a subject according to certain embodiments.

FIG. 6 shows a system, including a gene gun, for removing target genetic material from a subject according to certain embodiments.

FIG. 7 shows results from targeting an HPV genome using a targetable nuclease.

### DETAILED DESCRIPTION

The invention provides for the therapeutic administration of a gene-editing nuclease by methods that avoid an immune response or reduce intracellular toxicity mediated by protein-protein interactions. Specifically, gene-editing nucleases are introduced in a manner that avoids triggering an immune response that negates a therapeutic effect of the nuclease treatment. Preferably, the therapeutic effect is achieved through the administration of a plurality of nucleases that cleave the same target but that are not cross-immunogenic. In other words, the nucleases have the same therapeutic effect but do not trigger cross-reacting antibodies. The nucleases may differ by being: from different bacterial species; of different types (e.g., cas9, TALEN, ZFN, etc.); modified versions of a nuclease; or combinations thereof. In preferred embodiments, at least one nuclease from a CRISPR-Cas system is used.

CRISPR-Cas systems—originally microbial immune systems—use RNA-guided nucleases to cleave target nucleic acid. CRISPR systems include repetitive elements interspaced by short variable sequences derived from exogenous DNA targets known as protospacers (the crRNA) array. The Type II CRISPR includes the nuclease Cas9, the crRNA array that encodes the guide RNAs and a required auxiliary trans-activating crRNA (tracrRNA) that facilitates the processing of the crRNA array. The crRNA and tracrRNA can be fused as a single-guide RNA (sgRNA). Each crRNA unit then contains a 20-nt guide sequence that directs Cas9 to a 20-bp DNA target via Watson-Crick base pairing (a protospacer-adjacent motif (PAM) must appear adjacent the target). Different Cas9 orthologs may have different PAM requirements. The RNA-guided nuclease function of CRISPR-Cas may be applied in mammalian cells through delivery or heterologous expression of Cas9 and guide RNA (e.g., sgRNA or crRNA and tracrRNA). Cas9 may target almost any target of interest adjacent a PAM by altering the 20-nt guide sequence within the guide RNA. Besides Cas9, guided nucleases of the CRISPR-Cas type include Cas6, Cpf1, and modified versions any of those. Cas-type guided nucleases may be delivered as guide RNA and mRNA encoding nuclease, or as a ribonucleoprotein, or encoded in DNA sense, e.g., on a plasmid. A nuclease may be provided as an active protein or ribonucleoprotein, encoded in DNA or as an mRNA in pharmaceutical compositions of the invention. Compositions and methods of the invention reduce toxicity and immunogenicity of gene-editing systems through serial delivery of varied nucleases in multi-dose treatments; modification of a nuclease enzyme or guide RNA; ex vivo treatment followed by transplantation; and efficient treatment through careful measurement of pre- and post-treatment viral load (when targeting viral nucleic acids with the nuclease); and measurement of treatment byproducts such as proteins and on target and off target cuts of nucleic acid.

Methods include delivering a plurality of nucleases that cleave the same target but that are not cross-immunogenic. Any suitable plurality of nucleases that cleave the same target may be used. The different nucleases may be provided by, for example, progressively modified Cas9 nucleases, such as those discussed below, or by using different nucleases (e.g., Cas6, Cas9, and Cpf1), nucleases from different species, or some combination thereof across doses.

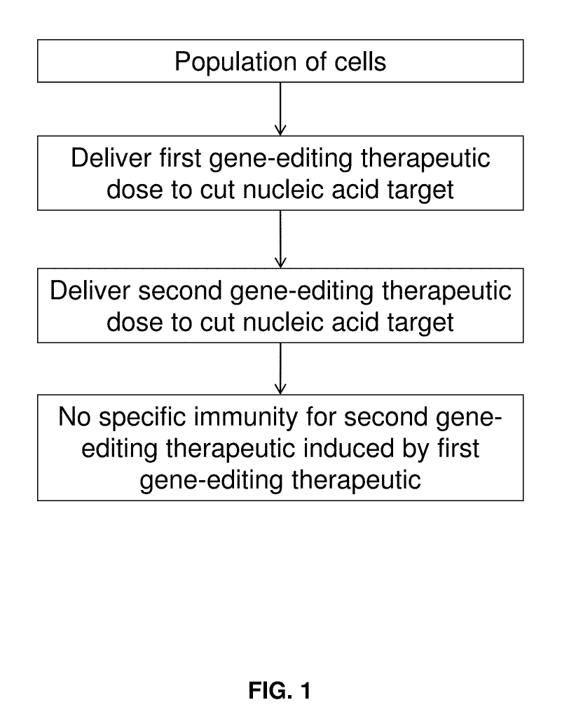

FIG. 1 diagrams methods of the invention. The method includes delivering, to a population of cells, a first nuclease to cut a nucleic acid target, and then delivering a second nuclease to cut the same nucleic acid target where the first nuclease does not induce specific immunity to the second nuclease in the population of cells.

Delivery schedules of the invention are designed to avoid priming and boosting an immune system against one or more of the nucleases being used. Priming and boosting refers to the development of specific immunity to an antigen through exposure to that antigen.

In cases of repeated dosing with a foreign protein, host cells may develop specific immunity to that protein, such that the immune system would clear subsequent doses of the protein, preventing its therapeutic use. By varying a nuclease from dose to dose, whether through modification or use of a different nuclease, a second or subsequent dose may avoid recognition by the cell's immune system which has been primed to respond to the first dose nuclease. Furthermore, because the cell's immune system has been primed for the first dose nuclease, the efficacy of the varied second dose may in fact be enhanced through reduction in immunogenicity. See Woodland, 2004, Jump-starting the immune system: prime-boosting comes of age, Trends Immunol 25(2):98, incorporated by reference.

Just as different nucleases are used in second and subsequent doses, different delivery vehicles may be used across doses. For example, a different or modified vector (e.g. viral vectors belonging to various serotypes) may be used to transfect the host cells with a nuclease targeting the same nucleic acid sequence for cutting. See, Bessis, et al., Immune responses to gene therapy vectors: influence on vector function and effector mechanisms, Gene Therapy (2004) 11, S10-S17, incorporated by reference.

Modified nucleases may include variants of Cas6, Cas9, or Cpf1 that differ by at least one peptide from the wild type counterpart nuclease but maintain wild type functionality. Modified nucleases may share sequence identity with the wild type counterpart of, for example, 70%, 80%, or 90% so long as guided nucleic acid cutting function is retained. Examples of modified nucleases compatible with delivery schedules of the invention are discussed below. In various embodiments, different doses may use Cas9 or other nucleases originating from different species. For example, the first and second doses may comprise Cas9 from 2 different species such as P. lavamentivorans, C. diphtheria, S. pasteurianus, N. cinerea, S. aureus, C. lari, S. pyogenes, and S. thermophilius. See, Ran et al., 2015, In vivo genome editing using Staphylococcus aureus Cas9, Nature, 520:186-191, incorporated by reference. These methods may be used in multi-dose therapies. In certain embodiments, treatment may be spread among multiple doses in order to reduce the amount of nuclease or other therapeutic introduced at any one time and, accordingly, reduce toxicity and immunogenicity. By varying the nuclease, used in each dose, or every few doses, immunogenicity may be further reduced. In various embodiments dosing schedules may include one or more doses a day, a week, a month, or multiple months. In certain embodiments, viral load and treatment efficacy may be assessed after each dose and the next dose modified accordingly. Certain embodiments of the invention relate to inducing specific tolerance to the nuclease or delivery vectors to be used in a therapy before the therapy. This may be accomplished through exposure to less immunogenic portions of the gene-editing system (e.g., exposure only to the guide RNA to be used in cases where the target nucleic acid is a native sequence). See, Bessis, 2004.

### Nuclease

Methods of the invention include using a nuclease to cleave a target nucleic acid without priming and boosting an immune system against subsequent treatments. Any suitable targeting nuclease can be used with methods of the invention including, for example, zinc-finger nucleases (ZFNs), transcription activator-like effector nucleases (TALENs), clustered regularly interspaced short palindromic repeat (CRISPR) nucleases, meganucleases, other endo- or exo-nucleases, or combinations thereof. See Schiffer, 2012, Targeted DNA mutagenesis for the cure of chronic viral infections, J Virol 88(17):8920-8936, incorporated by reference. In certain embodiments, progressive doses in a treatment regimen may use different targeting nucleases across the doses.

CRISPR methodologies employ a nuclease, CRISPR-associated (Cas9), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas9 and guide RNA (gRNA) may be synthesized by known methods. Cas9/guide-RNA (gRNA) uses a non-specific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11; each incorporated by reference.

In an aspect of the invention, Cas9 or a related Cas-type nuclease causes a break in a target nucleic acid such as a viral nucleic acid within cells of a human subject. This prevents the virus from replicating or re-entering an active, virulent stage of infection, and thus clears the host cell of the viral infection.

In embodiments of the invention, nucleases cleave the genome of the target virus. A nuclease is an enzyme capable of cleaving the phosphodiester bonds between the nucleotide subunits of nucleic acids. Nucleases are enzymes that cleave the phosphodiester bond within a polynucleotide chain. Some, such as Deoxyribonuclease I, cut DNA relatively nonspecifically (without regard to sequence), while many, typically called restriction nucleases or restriction enzymes, cleave only at very specific nucleotide sequences. In a preferred embodiment of the invention, the Cas9 nuclease is incorporated into the compositions and methods of the invention, however, it should be appreciated that any nuclease may be utilized.

In preferred embodiments of the invention, the Cas9 nuclease is used to cleave the target nucleic acid in at least one treatment dose. The Cas9 nuclease is capable of creating a double strand break in the genome. The Cas9 nuclease has two functional domains: RuvC and HNH, each cutting a different strand. When both of these domains are active, the Cas9 causes double strand breaks in the genome.

In some embodiments of the invention, insertions into the genome can be designed to cause incapacitation, or altered genomic expression. Additionally, insertions/deletions are also used to introduce a premature stop codon either by creating one at the double strand break or by shifting the reading frame to create one downstream of the double strand break. Any of these outcomes of the NHEJ repair pathway can be leveraged to disrupt the target gene. The changes introduced by the use of a Cas9 may be permanent.

In some embodiments of the invention, at least one insertion is caused by a nuclease. In a preferred embodiment, numerous insertions are caused in the genome, thereby incapacitating the virus. In an aspect of the invention, the number of insertions lowers the probability that the genome may be repaired.

In some embodiments of the invention, at least one deletion is caused by a nuclease. In a preferred embodiment, numerous deletions are caused in the genome, thereby incapacitating the virus. In an aspect of the invention, the number of deletions lowers the probability that the genome may be repaired. In a highly-preferred embodiment, nucleases of the invention cause significant disruption of viral nucleic acid, resulting in effective destruction of the viral genome, while leaving the host genome functional.

TALENs use a nonspecific DNA-cleaving nuclease fused to a DNA-binding domain that can be to target essentially any sequence. For TALEN technology, target sites are identified and expression vectors are made. Linearized expression vectors (e.g., by NotI) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55, incorporated by reference.

TALEN and CRISPR methods provide one-to-one relationship to the target sites, i.e. one unit of the tandem repeat in the TALE domain recognizes one nucleotide in the target site, and the crRNA, gRNA, or sgRNA of CRISPR/Cas system hybridizes to the complementary sequence in the DNA target. Methods can include using a pair of TALENs or a Cas9 protein with one gRNA to generate double-strand breaks in the target. The breaks are then repaired via non-homologous end-joining or homologous recombination (HR).

ZFN may be used to cut viral nucleic acid. Briefly, the ZFN method includes introducing into the infected host cell a ZFN or a vector (e.g., plasmid) encoding a targeted ZFN 305 and, optionally, at least one accessory polynucleotide. See, e.g., U.S. Pub. 2011/0023144 to Weinstein, incorporated by reference. The cell includes target sequence. The cell is incubated to allow expression of the ZFN, wherein a double-stranded break is introduced into the targeted sequence by the ZFN. In some embodiments, a donor polynucleotide or exchange polynucleotide is introduced. Swapping a portion of the viral nucleic acid with irrelevant sequence can fully interfere transcription or replication of the viral nucleic acid. Target DNA along with exchange polynucleotide may be repaired by an error-prone non-homologous end-joining DNA repair process or a homology-directed DNA repair process.

Typically, a ZFN comprises a DNA binding domain (i.e., zinc finger) and a cleavage domain (i.e., nuclease) and this gene may be introduced as mRNA (e.g., 5′ capped, polyadenylated, or both). Zinc finger binding domains may be engineered to recognize and bind to any nucleic acid sequence of choice. See, e.g., Qu et al., 2013, Zinc-finger-nucleases mediate specific and efficient excision of HIV-1 proviral DAN from infected and latently infected human T cells, Nucl Ac Res 41(16):7771-7782, incorporated by reference. The cleavage domain portion of the ZFNs may be obtained from any suitable nuclease or exonuclease such as restriction nucleases and homing nucleases. See, for example, Belfort & Roberts, 1997, Homing nucleases: keeping the house in order, Nucleic Acids Res 25(17):3379-3388, incorporated by reference. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction nucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. No. 5,356,802; U.S. Pat. No. 5,436,150; U.S. Pat. No. 5,487,994; U.S. Pub. 2005/0064474; U.S. Pub. 2006/0188987; and U.S. Pub. 2008/0131962, each incorporated by reference.

Meganucleases are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into families based on sequence and structure motifs. The families include GIY-YIG, HNH, His-Cys box and PD-(D/E)XK. Meganucleases have been found in all kingdoms of life, generally encoded within introns or inteins although freestanding members also exist. These nucleases are characterized by a protein motif essential for enzymatic activity. Some proteins contained only one such motif, while others contained two; in both cases the motifs were followed by ˜75-200 amino acid residues having little to no sequence similarity with other family members. Crystal structures illustrates mode of sequence specificity and cleavage mechanism for the these nucleases: (i) specificity contacts arise from the burial of extended β-strands into the major groove of the DNA, with the DNA binding saddle having a pitch and contour mimicking the helical twist of the DNA; (ii) full hydrogen bonding potential between the protein and DNA is never fully realized; (iii) cleavage to generate the characteristic 4-nt 3′-OH overhangs occurs across the minor groove, wherein the scissile phosphate bonds are brought closer to the protein catalytic core by a distortion of the DNA in the central “4-base” region; (iv) cleavage occurs via a proposed two-metal mechanism, sometimes involving a unique “metal sharing” paradigm; (v) and finally, additional affinity and/or specificity contacts can arise from “adapted” scaffolds, in regions outside the core α/β fold. See Silva et al., 2011, Meganucleases and other tools for targeted genome engineering, Curr Gene Ther 11(1):11-27, incorporated by reference.

Some embodiments of the invention may utilize modified version of a nuclease. Modified versions of the Cas9 enzyme containing a single inactive catalytic domain, either RuvC− or HNH−, are called ‘nickases’. With only one active nuclease domain, the Cas9 nickase cuts only one strand of the target DNA, creating a single-strand break or ‘nick’. Similar to the inactive dCas9 (RuvC− and HNH−), a Cas9 nickase is still able to bind DNA based on gRNA specificity, though nickases will only cut one of the DNA strands. The majority of CRISPR plasmids are derived from S. pyogenes and the RuvC domain can be inactivated by a D10A mutation and the HNH domain can be inactivated by an H840A mutation.

A single-strand break, or nick, is normally quickly repaired through the HDR pathway, using the intact complementary DNA strand as the template. However, two proximal, opposite strand nicks introduced by a Cas9 nickase are treated as a double strand break, in what is often referred to as a ‘double nick’ or ‘dual nickase’ CRISPR system. A double-nick induced double strain break can be repaired by either NHEJ or HDR depending on the desired effect on the gene target. At these double strain breaks, insertions and deletions are caused by the CRISPR/Cas9 complex. In an aspect of the invention, a deletion is caused by positioning two double strand breaks proximate to one another, thereby causing a fragment of the genome to be deleted.

One goal of modifying nucleases to decrease immunogenicity includes making the nuclease smaller. Structural analysis has identified a conserved structural core across Cas9 proteins with variable regions appended thereto which have been speculated to relate to guide structure recognition. See Jinek, et al., Structures of Cas9 Nucleases Reveal RNA-Mediated Conformational Activation, Science. 2014 Mar. 14; 343(6176): 1247997, incorporated by reference. In certain embodiments, modified Cas9 proteins may include removal of extraneous structures that do not contribute to functionality of the Cas9 complex in order to provide a smaller protein or plasmid for delivery into a cell to be treated. In various embodiments, mutational screening may be conducted on Cas9 variants to determine minimum structure to achieve desired functionality.

For example, a plasmid coding SpCas9 and guide RNA against GFP may be used as the starting material. A plasmid library with different deletions around the potential redundant loop or other extraneous structure can be delivered to 293T cells with single copy GFP gene integrated into the genome. After 3 days incubation, cells with low GFP signals are harvested for DNA isolation. The Cas9 coding sequences in these cells can then be sequenced to reveal the particular deletions. Such experiments can be used with various guide RNAs to develop application specific minimally sized functional complexes for specific applications.

Cas9 may be modified to alter catalytic function, guide RNA specificity, or protospacer adjacent motif requirements. Id.

In certain embodiments, Cas9 may be included in a fusion protein. Proteins or portions thereof can be added to Cas9 in a fusion to, for example, change the charge and/or hydrophobicity (e.g., by adding GFP) which may affect immunogenicity. The charge and/or hydrophobicity may alternatively be changed through alteration of amino acid residues at locations that do not effect folding from a neutral residue or a residue with one charge and/or hydrophobicity to a residue with another charge and/or hydrophobicity. Fusion proteins can also be created with Cas9 and other proteins or portions thereof that carry a different immune profile in order to reduce immunogenicity.

In certain embodiments, the immune system can be used to aid delivery of Cas9-type therapies. See, Wu & Wu, 1987, Receptor-mediated in vitro gene transformation by a soluble DNA carrier system, J Biol Chem 262:4429; Rojanasakul et al., 1994, Targeted gene delivery to alveolar macrophages via Fc receptor-mediated endocytosis, Pharm Res 11(12):1731-6; or Gupta et al., Single chain Fv: a ligand in receptor-mediated gene delivery, Gene Ther. 2001 April; 8(8):586-92; each incorporated by reference. Fusion proteins of the invention may include Fc/Cas9 fusions for Fc mediated uptake of the Cas9 (e.g., a protein or a nucleic acid that encodes a protein that is Fc+Cas9).

In some embodiments fusion proteins may be used to enhance tissue specific targeting which may reduce the amount of compound needed for successful treatment and reduce systemic distribution by keeping the compound localized at a target tissue. Both of these effects may reduce overall immunogenicity and toxicity. IgG or other proteins or antibodies could be used with Cas9 to target specific tissues. See Carter, Introduction to current and future protein therapeutics: A protein engineering perspective, Experimental Cell Research 317 (2011) 1261-1269. In various embodiments the fusion may also include a linker between Cas9 and the other protein or portion thereof. An albumin fusion may be used to increase plasma half-life of the compound while various cell-penetrating peptides may be used to aid delivery of the nuclease to the target cell.

In certain embodiments, Cas9 proteins from different bacterial or archaeal species may be used having distinguishable protospacer adjacent motif (PAM) requirements and nuclease activity. While the best-characterized Streptococcus pyogenes cas9 (SpCas9) offers wide target selections and high activity, it has some drawbacks for certain applications. For example, its large size represents a great challenge for delivery. The widely used AAV vectors for in vivo delivery of DNA have a payload capacity of only 4.5 kb. The small packaging capacity prevents the co-delivery of SpCas9 and guide RNA in the same vector. Many bacterial and archaeal species code for a 25% smaller cas9 protein. See, Jinek M, et al. Structures of Cas9 nucleases reveal RNA-mediated conformational activation, Science, 343(6176), 1247997; Ran, et al., In vivo genome editing using Staphylococcus aureus Cas9, Nature, 520, 186-191 (9 Apr. 2015); each incorporated by reference. Use of a smaller Cas9 protein, much like using a structurally modified, smaller, Cas9 protein, could enable the use of a smaller delivery compound reducing toxicity and immune response or allow for a longer targeting sequence, increasing efficiency and decreasing the needed amount of compound to achieve therapeutic effect.

In certain embodiments, gene-editing systems, such as nucleases discussed herein, may be humanized by reshaping regions to mimic human derived proteins. See, Cox, et al., Therapeutic Genome Editing: Prospects and Challenges, Nat Med. 2015 February; 21(2): 121-131; Riechmann, et al., Reshaping human antibodies for therapy, Nature 1988 Mar. 24; 332(6162): 323-7; Kolbinger, et al., Humanization of a mouse anti-human IgE antibody: a potential therapeutic for IgE-mediated allergies, Protein Eng. 1993 November; 6(8): 971-80.

In certain embodiments, in vitro random mutagenesis may be used to generate and identify functional nucleases such as Cas9 analogs which may be useful in various techniques disclosed herein such as variable Cas9 treatment delivery schedules. Random mutagenesis can be achieved by treating DNA or whole bacteria with various chemical mutagens, by passing cloned genes through mutator strains, by “error-prone” PCR mutagenesis, by rolling circle error-prone PCR, or by saturation mutagenesis. See, Labrou, Random mutagenesis methods for in vitro directed enzyme evolution, Curr Protein Pept Sci, 2010 February; 11(1):91-100, incorporated by reference.

### Targeting Sequence

In various embodiments, a nuclease may use the targeting specificity of a guide RNA (gRNA). As discussed below, guide RNAs or single guide RNAs are specifically designed to target a nucleic acid sequence to be cut by the nuclease (e.g., a virus genome). As used herein targeting sequence can mean any combination of gRNA, crRNA, tracrRNA, sgRNA, and others. A CRISPR/Cas9 gene editing complex of the invention works optimally with a guide RNA that targets the viral genome. Guide RNA (gRNA) (which includes single guide RNA (sgRNA), crisprRNA (crRNA), transactivating RNA (tracrRNA), any other targeting oligo, or any combination thereof) leads the nuclease to the viral genome in order to cause viral genomic disruption. In an aspect of the invention, CRISPR/Cas9/gRNA complexes are designed to target specific viruses within a cell. It should be appreciated that any virus can be targeted using the composition of the invention. Identification of specific regions of the virus genome aids in development and designing of CRISPR/Cas9/gRNA complexes.

In certain embodiments, the specificity of a guide RNA may be increased by using a longer sequence. Because overall size of the CRISPR/Cas9/gRNA complex is a limiting factor in successful introduction into a target cell, if the nuclease size can be reduced using any of the modifications discussed above, then the complex can tolerate a longer guide RNA. In certain embodiments, the gRNA target recognition sequence may be 20 mer, 21 mer, 22 mer, 23 mer, 24 mer, 25 mer, 26 mer, 27 mer, 28 mer, 29 mer, or 30 mer recognition sequence. Increasing specificity of the complex can increase treatment efficiency allowing for lower amounts of therapeutics to be used in treatment. By administering lower treatment volumes to a patient or ex vivo cells and tissue, toxicity and immunogenicity can also be reduced as there is less compound present in a patient to elicit a response.

In an aspect of the invention, the CRISPR/Cas9/gRNA complexes are designed to target latent viruses within a cell. Once transfected within a cell, the CRISPR/Cas9/gRNA complexes cause repeated insertions or deletions to render the genome incapacitated, or due to number of insertions or deletions, the probability of repair is significantly reduced.

As an example, we inactivated the Epstein-Barr virus (EBV), also called human herpesvirus 4 (HHV-4) in cells using a CRISPR/Cas9/gRNA complex. EBV is a virus of the herpes family, and is one of the most common viruses in humans. The virus is approximately 122 nm to 180 nm in diameter and is composed of a double helix of DNA wrapped in a protein capsid. In this example, the Raji cell line serves as an appropriate in vitro model. The Raji cell line is the first continuous human cell line from hematopoietic origin and cell lines produce an unusual strain of Epstein-Barr virus while being one of the most extensively studied EBV models. To target the EBV genomes in the Raji cells, a CRISPR/Cas9 complex with specificity for EBV is needed.

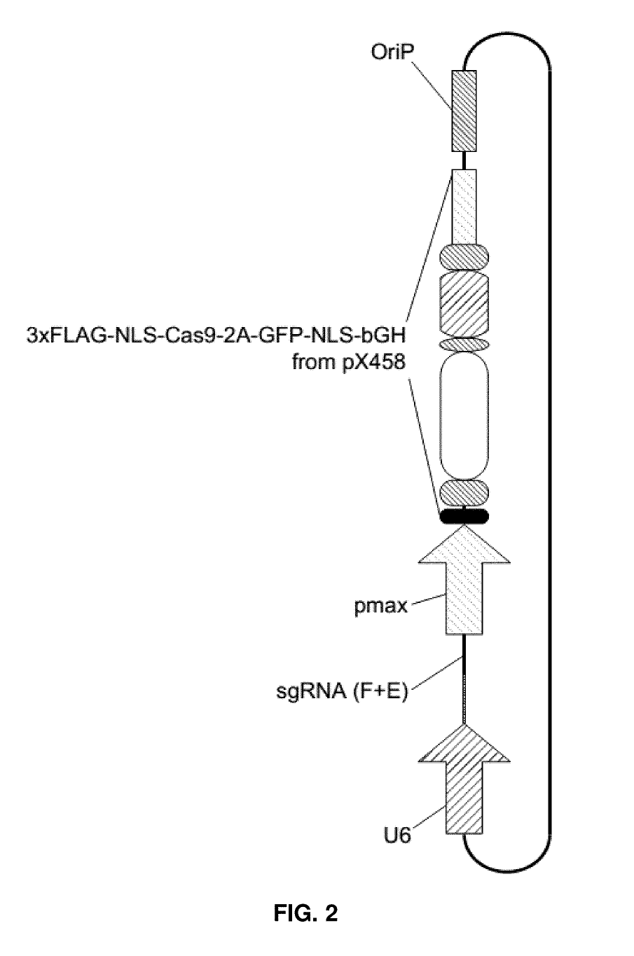

FIG. 2 shows a composition that includes an EGFP marker fused after the Cas9 protein. The design of EBV-targeting CRISPR/Cas9 plasmids consisting of a U6 promoter driven chimeric guide RNA (sgRNA) and a ubiquitous promoter driven Cas9 that were obtained from Addgene, Inc. Commercially available guide RNAs and Cas9 nucleases may be used with the present invention. The EGFP marker fused after the Cas9 protein allowed selection of Cas9-positive cells.

Guide RNAs may be designed, for example, to target a specific part of an HPV genome. The target area in HPV is identified and guide RNA to target selected portions of the HPV genome are developed and incorporated into the composition of the invention. In an aspect of the invention, a reference genome of a particular strain of the virus is selected for guide RNA design.

In relation to EBV, for example, the reference genome from strain B95-8 was used as a design guide. Within a genome of interest, such as EBV, selected regions, or genes are targeted. For example, six regions can be targeted with seven guide RNA designs for different genome editing purposes.

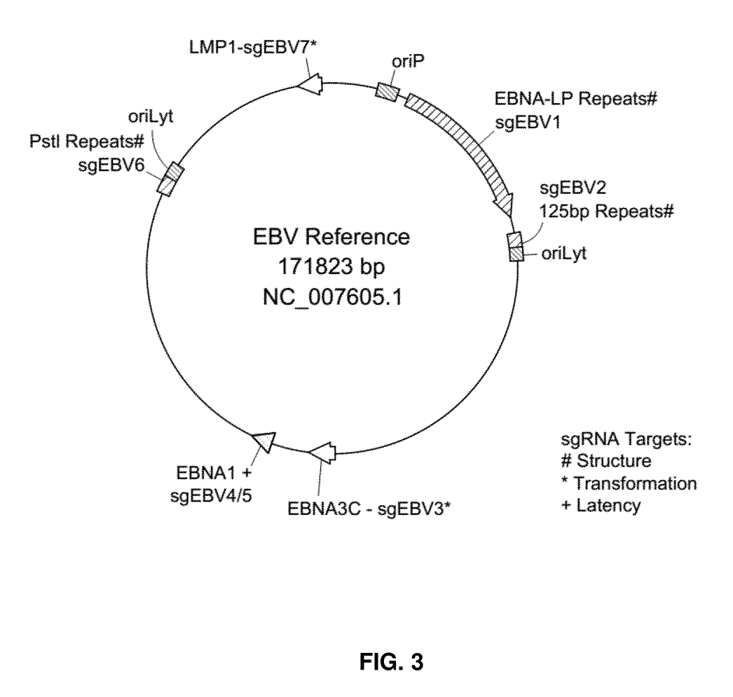

FIG. 3 shows gRNA targets along a reference genome where # denotes structural targets, * denotes transformation-related targets, and + denotes latency-related targets.

In relation to EBV, EBNA1 is the only nuclear Epstein-Barr virus (EBV) protein expressed in both latent and lytic modes of infection. While EBNA1 is known to play several important roles in latent infection, EBNA1 is crucial for many EBV functions including gene regulation and latent genome replication. Therefore, guide RNAs sgEBV4 and sgEBV5 were selected to target both ends of the EBNA1 coding region in order to excise this whole region of the genome. These “structural” targets enable systematic digestion of the EBV genome into smaller pieces. EBNA3C and LMP1 are essential for host cell transformation, and guide RNAs sgEBV3 and sgEBV7 were designed to target the 5′ exons of these two proteins respectively.

### Quantification of Treatment and Effects

As discussed above, overall toxicity and immunogenicity can be reduced by lowering the amount of nuclease/gRNA complex present or needed in the host body or tissue. Methods of the invention advantageously reduce the amount of complex needed in treatment through careful measurement of the amount of complex being administered, the build-up of treatment byproducts, and pre and post-treatment viral load to tailor treatment amounts and judge efficacy to determine end points.

In certain embodiments, methods of the invention relate to measuring treatment amounts. One problem with existing techniques is that measurement of delivered compound will systematically underestimate the amount of foreign material actually introduced. Current measurement techniques involve using PCR to amplify and sequence a product of successful treatment by Cas9. These methods do not account for the full amount of compound delivered because it fails to take into account the products of un-successful Cas9 treatment (e.g., Cas9 that did not reach a target or did cut a nucleic acid off target). Accordingly, these measured amounts do not fully capture the amount of compound present in the tissue or body which may be eliciting an immune response or reaching toxic levels.

In certain embodiments, protein products may be measured. This can be accomplished through any known method including by using flow cytometry, antibodies, or a global analysis such as Eliza. In certain embodiments, PCR may be used to measure viral load but with additional primers to measure related targets to better capture both expected cut nucleic acid as well as off target products, providing a more accurate understanding of the amount of foreign material that has been introduced into the tissue or body. In some embodiments, host proteins affected by cut DNA products may be monitored to more accurately reflect the amount of foreign material being introduced by treatment.

In certain embodiments, accurate determination of viral load may be used to better tailor the amount of compound used to the amount of viral DNA to be cut. Correctly measuring viral load may allow an improved immunogenicity profile because it will aid in delivering the correct amount of the treatment. See, Puren et al., 2010, Laboratory Operations, Specimen Processing, and Handling for Viral Load Testing and Surveillance, J Inf Dis 201:S27-36), incorporated by reference. Additionally, not all viral DNA present in a cell may require treatment. For example, evidence suggests that as much as 90% of Epstein Barr viral DNA in a cell or in tissue may not be live infection. Accordingly, treatment amounts should be tailored to the live virus to be treated thereby reducing compound levels and associated immune response and toxicity.

In various embodiments, viral load may be measured at regular points during treatment while conservative amounts of compound are administered so that treatment amounts can be adjusted up if needed and stopped when target levels have been achieved. Excessive levels of compound can thereby be avoided, reducing the chance of immune response or toxicity.

### Delivery Methods

Methods of the invention include introducing a nuclease and a sequence-specific targeting moiety to a target cell (e.g. an infected cell). In order to achieve effective treatment across a variety of cell types (e.g., treatment of a mixed population of cells), both the gene-editing system and the delivery method for introducing the gene-editing system into the cell must not damage cell viability. In cases of treating an infected cell, the nuclease may be targeted to the viral nucleic acid by means of the sequence-specific targeting moiety where it then cleaves the viral nucleic acid without interfering with a host genome. Any suitable method can be used to deliver the nuclease to the infected cell or tissue. In certain embodiments, delivery method may be tailored to the cell type to be treated and the treatment setting (e.g., in vivo or ex vivo). For example, the nuclease or the gene encoding the nuclease may be delivered by injection, orally, or by hydrodynamic delivery. The nuclease or the gene encoding the nuclease may be delivered to systematic circulation or may be delivered or otherwise localized to a specific tissue type. The nuclease or gene encoding the nuclease may be modified or programmed to be active under only certain conditions by using, for example, a tissue-specific promoter so that the encoded nuclease is preferentially or only transcribed in certain tissue types. In certain embodiments, such as with in vitro delivery to extracted cells (e.g., hematopoietic stem cells from a patient's bone marrow), cell survival is extremely important and transfection techniques such as electroporation may be less desirable for their potential to harm the cell. Cellular deliver methods contemplated by the invention include the use of adenoviruses as described below as well as clonal micelles and copolymer blocks. See, Zhang, et al., Gene transfection in complex media using PCBMAEE-PCBMA copolymer with both hydrolytic and zwitterionic blocks, Biomaterials. 2014 September; 35(27):7909-18, incorporated by reference.

In some embodiments, a cocktail of guide RNAs may be introduced into a cell. The guide RNAs are designed to target numerous categories of sequences of the viral genome. By targeting several areas along the genome, the double strand break at multiple locations fragments the genome, lowering the possibility of repair. Even with repair mechanisms, the large deletions render the virus incapacitated.

In some embodiments, several guide RNAs are added to create a cocktail to target different categories of sequences. For example, two, five, seven or eleven guide RNAs may be present in a CRISPR cocktail targeting three different categories of sequences. However, any number of gRNAs may be introduced into a cocktail to target categories of sequences. In preferred embodiments, the categories of sequences are important for genome structure, host cell transformation, and infection latency, respectively.

In some aspects of the invention, in vitro experiments allow for the determination of the most essential targets within a viral genome. For example, to understand the most essential targets for effective incapacitation of a genome, subsets of guide RNAs are transfected into model cells. Assays can determine which guide RNAs or which cocktail is the most effective at targeting essential categories of sequences.

For example, in the case of the EBV genome targeting, seven guide RNAs in the CRISPR cocktail targeted three different categories of sequences which are identified as being important for EBV genome structure, host cell transformation, and infection latency, respectively. To understand the most essential targets for effective EBV treatment, Raji cells were transfected with subsets of guide RNAs. Although sgEBV4/5 reduced the EBV genome by 85%, they could not suppress cell proliferation as effectively as the full cocktail. Guide RNAs targeting the structural sequences (sgEBV1/2/6) could stop cell proliferation completely, despite not eliminating the full EBV load (26% decrease). Given the high efficiency of genome editing and the proliferation arrest, it was suspect that the residual EBV genome signature in sgEBV1/2/6 was not due to intact genomes but to free-floating DNA that has been digested out of the EBV genome, i.e. as a false positive.

Aspects of the invention allow for nucleases to be delivered to cells by various methods, including viral vectors and non-viral vectors. Viral vectors may include retroviruses, lentiviruses, adenoviruses, and adeno-associated viruses. It should be appreciated that any viral vector may be used to deliver a nuclease. The vectors may contain essential components such as origin of replication, e.g., for the replication and maintenance of the vector in the host cell.

In an aspect of the invention, viral vectors are used as delivery vectors to deliver the nucleases into a cell. Use of viral vectors as delivery vectors are known in the art. See for example U.S. Pub. 2009/0017543 to Wilkes et al., incorporated by reference.

Retroviral vectors may be used to introduce nucleic acids into a cell. A retrovirus is a single-stranded RNA virus that stores its nucleic acid in the form of an mRNA genome (including the 5′ cap and 3′ PolyA tail) and targets a host cell as an obligate parasite. Once inside the host cell cytoplasm the virus uses its own reverse transcriptase enzyme to produce DNA from its RNA genome. This new DNA may be incorporated into the host cell genome by an integrase enzyme, at which point the retroviral DNA is referred to as a provirus. For example, the recombinant retroviruses such as the Moloney murine leukemia virus have the ability to integrate into the host genome in a stable fashion. They contain a reverse transcriptase that allows integration into the host genome. Retroviral vectors can either be replication-competent or replication-defective. In some embodiments of the invention, retroviruses are used to deliver nucleases.

In some embodiments of the invention, lentiviruses, which are a subclass of retroviruses, are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV may be used as delivery vectors since they do not integrate into the host's genome. In some aspects of the invention, only the viral genome to be targeted is effected by the CRISPR/Cas9/gRNA complexes, and not the host's cells. Adeno-associated virus (AAV) is a small virus that infects humans and some other primate species. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Additionally or alternatively, methods and compositions of the invention may use herpesvirus, poxvirus, alphavirus, or vaccinia virus as a means of delivery vectors.

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors based on charged lipids or polymers can complex with charged nucleic acids, proteins, or ribonucleoproteins to form particles with a diameter in the order of 100 nm. Alternatively, synthetic vectors can complex with nucleic acids, proteins, or ribonucleoproteins based on complementary hydrophobicity to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous administration. PEGylated nanoparticles are therefore often referred as “stealth” nanoparticles. The nanoparticles that are not rapidly cleared from the circulation will have a chance to encounter infected cells. However, PEG on the surface can decrease the uptake by target cells and reduce the biological activity. Therefore, to attach targeting ligand to the distal end of the PEGylated component is necessary; the ligand is projected beyond the PEG “shield” to allow binding to receptors on the target cell surface.

When a cationic liposome is used as gene carrier, the application of neutral helper lipid is helpful for the release of nucleic acid, besides promoting hexagonal phase formation to enable endosomal escape. In some embodiments of the invention, neutral or anionic liposomes are developed for systemic delivery of nucleic acids and obtaining therapeutic effect in experimental animal model. Designing and synthesizing novel cationic lipids and polymers, and covalently or non-covalently binding gene with peptides, targeting ligands, polymers, or environmentally sensitive moieties also attract many attentions for resolving the problems encountered by non-viral vectors. The application of inorganic nanoparticles (for example, metallic nanoparticles, iron oxide, calcium phosphate, magnesium phosphate, manganese phosphate, double hydroxides, carbon nanotubes, and quantum dots) in delivery vectors can be prepared and surface-functionalized in many different ways.

In some embodiments, nucleases are delivered using nanoparticles or nanosystems. Nanoparticles, such as liposomes, albumin-based particles, PEGylated proteins, biodegradable polymer-drug composites, polymeric micelles, dendrimers, among others, may be beneficial for systemic delivery. See Davis et al., 2008, Nanotherapeutic particles: an emerging treatment modality for cancer, Nat Rev Drug Discov. 7(9):771-782, incorporated by reference. Long circulating macromolecular carriers such as liposomes, can exploit the enhanced permeability and retention effect for preferential extravasation from tumor vessels. In certain embodiments, nucleases of the invention or their vectors are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70, incorporated by reference.

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component; each incorporated by reference.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

The delivery vector may be selected, varied, or modified to minimize an immune response or otherwise optimize delivery of the nucleases. For example, when targeting EBV, since lymphocytes are known for being resistant to lipofection, nucleofection (a combination of electrical parameters generated by a device called Nucleofector, with cell-type specific reagents to transfer a substrate directly into the cell nucleus and the cytoplasm) was necessitated for DNA delivery into the Raji cells. The Lonza pmax promoter drives Cas9 expression as it offered strong expression within Raji cells. 24 hours after nucleofection, obvious EGFP signals were observed from a small proportion of cells through fluorescent microscopy. The EGFP-positive cell population decreased dramatically, however, <10% transfection efficiency 48 hours after nucleofection was measured.

Any suitable delivery pathway may be used to deliver nucleases to cells while avoiding an immune response. Common known pathways include transdermal, transmucal, nasal, ocular and pulmonary routes. Drug delivery systems may include liposomes, proliposomes, microspheres, gels, prodrugs, cyclodextrins, etc. Aspects of the invention utilize nanoparticles composed of biodegradable polymers to be transferred into an aerosol for targeting of specific sites or cell populations in the lung, providing for the release of the drug in a predetermined manner and degradation within an acceptable period of time. Controlled-release technology (CRT), such as transdermal and transmucosal controlled-release delivery systems, nasal and buccal aerosol sprays, drug-impregnated lozenges, encapsulated cells, oral soft gels, iontophoretic devices to administer drugs through skin, and a variety of programmable, implanted drug-delivery devices are used in conjunction with the complexes of the invention of accomplishing targeted and controlled delivery.

To deliver nucleases, the invention provides for the use various methods to increase permeability of the target tissue and control uptake of the therapeutic compound. These delivery methods may include one or more of the following, ultrasound mediated delivery (both high and low frequency or cavitational or non-cavitational), iontophoretic transdermal delivery, electroporation, chemical mediated delivery, thermal ablation of the stratum corneum, magnetophoresis, photomechanical waves, and mechanical methods such as microdermabrasion and microneedles. See Prausnitz & Langer, Transdermal drug delivery, Nature Biotechnology 26, 1261-1268 (2008), the contents of which are incorporated herein in their entirety for all purposes. Many of the above methods include applications in transdermal delivery across the stratum corneum as well as delivery across intracellular delivery by inducing cell membrane fluidity and allowing nucleic acid compositions of the invention to pass into cells.

In various embodiments, permeability enhancing energy may be delivered to cells or tissue through ultrasound waves. See Smith, Perspectives on transdermal ultrasound mediated drug delivery, Int J Nanomedicine. 2007 December; 2(4): 585-594, the contents of which are incorporated herein in their entirety for all purposes. These methods are sometimes referred to a sonophoresis or phonophoresis. Ultrasound mediated transdermal drug delivery may be used with a range of ultrasound frequencies and is generally categorized as high frequency (e.g., around 1-3 MHz) or low frequency (e.g., around 20 kHz). Ultrasound mediated transdermal drug delivery is sometimes divided into cavitational and noncavitational methods. Low frequency ultrasound is generally more effective at enhancing transdermal drug transport through cavitation induced bilayer disordering of the stratum corneum. Id. The permeability effects of cavitational bubbles generated in the stratum corneum through low frequency ultrasound may last for many hours. Prausnitz, 2008.

Ultrasound may be used to facilitate passage of compounds across cellular membranes in the form of encapsulated ultrasound microbubbles in any tissue. See Nozaki, et al., Enhancement of ultrasound-mediated gene transfection by membrane modification, The Journal of Gene Medicine, Vol. 5, Issue 12, pp. 1046-1055, December 2003; Liu, et al., Encapsulated ultrasound microbubbles: Therapeutic application in drug/gene delivery, Journal of Controlled Release, Vol. 114, Issue 1, 10 Aug. 2006, pp. 89-99; the contents of each which are incorporated herein in their entirety and for all purposes. Low-intensity ultrasound in combination with microbubbles has recently acquired much attention as a safe method of gene delivery. Ultrasound shows tissue-permeabilizing effect. It is non-invasive and site-specific. Ultrasound-mediated microbubbles have been proposed as an innovative method for noninvasive delivery of drugs and nucleic acids to different tissues. In ultrasound-triggered drug delivery, tissue-permeabilizing effect can be potentiated using ultrasound contrast agents, gas-filled microbubbles. The use of microbubbles for delivery of nucleic acids is based on the hypothesis that destruction of DNA-loaded microbubbles by a focused ultrasound beam during their microvascular transit through the target area will result in localized transduction upon disruption of the microbubble shell while sparing non-targeted areas. See Tsutsui et al., 2004, The use of microbubbles to target drug delivery, Cardiovasc Ultrasound 2:23, the contents of which are incorporated by reference.

Small, lipophilic compounds may be delivered with noncavitational ultrasound but success is limited with other, larger compounds. Heat has been shown to enhance transdermal delivery of some compounds and one aspect of ultrasound mediated delivery is the generation of heat in the tissue by the ultrasound waves.

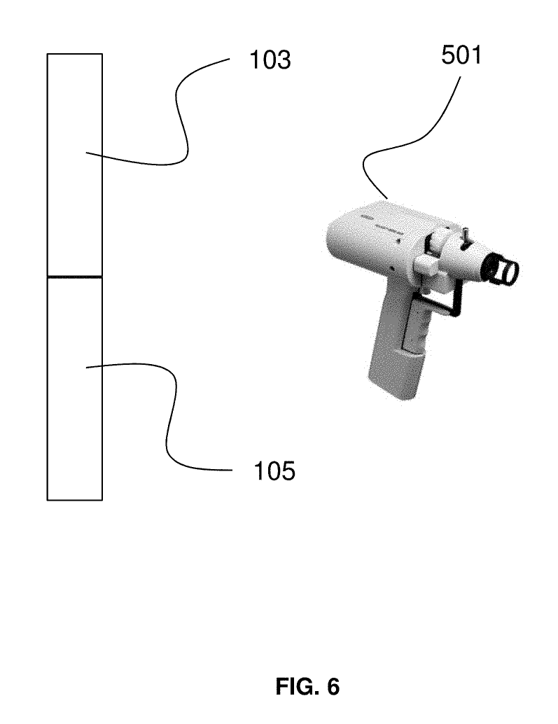

Ultrasound waves may be applied using single element or other known types of transducers such as those available from Blatek, Inc. (State College, Pa.). Thus, in some embodiments, the invention provides a system for treating a viral infection that includes an ultrasound transducer 301, a vector encoding a gene for an enzyme that cuts target genetic material such as Cas9 103, and a gRNA that targets a latent virus and that has no match in the human genome 105, as shown in FIG. 6.

In certain embodiments, transdermal delivery may be enhanced through electroporation of the skin tissue. See Prausnitz, et al., Electroporation of mammalian skin: A mechanism to enhance transdermal drug delivery, Proc. Natl. Acad. Sci. USA Vol. 90, pp. 10504-10508, November 1993, the contents of which are incorporated herein in their entirety for all purposes.

Electroporation involves the use of short, high-voltage pulses of electricity to reversibly disrupt cell membranes. Electroporation, like cavitational ultrasound, disrupts lipid bilayer structures in the skin, allowing for increased permeability and, accordingly, enhanced drug delivery. The electropores created through electroporation can persist for hours after treatment, and transdermal transport can be increased by orders of magnitude for small molecule drugs, peptides, vaccines and DNA. Side effects of electroporation, such as pain and muscle stimulation from the nerves below the stratum corneum layer, can be minimized through the use of closely spaced microelectrodes to constrain the electric field within the stratum corneum. Prausnitz, 2008.

Electroporation of cellular membranes can be used to increase cell membrane fluidity and allow passage of compounds into individual cells. See Ho, et al., Electroporation of Cell Membranes: A Review, Critical Reviews in Biotechnology, Vol. 16, Issue 4, 1996; Zhang, et al., Development of an Efficient Electroporation Method for Iturin A-Producing Bacillus subtilis ZK, Int. J. Mol. Sci. 2015, 16, 7334-7351; the contents of each which are incorporated herein in their entirety and for all purposes. Electroporation of cell membranes uses the same principles as described above with respect to transdermal applications. Id. As cell viability is essential to the methods of the invention, care must be taken in the application of the short high-voltage pulses.

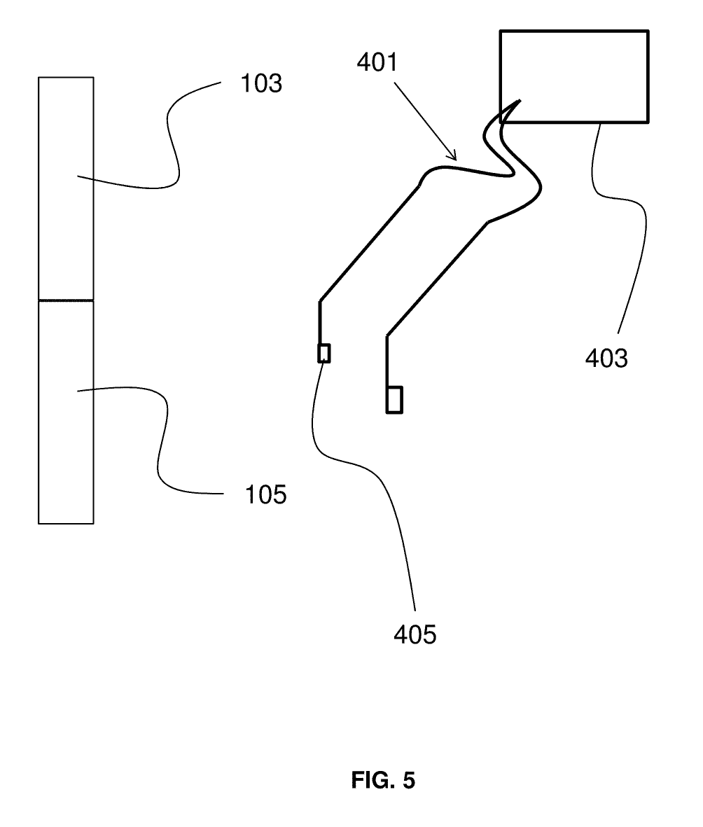

Electroporation may be performed using an electroporation device 401 comprising, for instance, an electroporation generator 403 and electrodes 405 such as the Gemini X2 system available from Harvard Apparatus, Inc. (Holliston, Mass.). Thus, in some embodiments, the invention provides a system for treating a viral infection that includes electroporation device 401 comprising an electroporation generator 403 and electrodes 405, a vector encoding a gene for an enzyme that cuts target genetic material such as Cas9 103, and a gRNA that targets a latent virus and that has no match in the human genome 105, as shown in FIG. 5.

In various embodiments nucleic acid compositions of the invention may be introduced into host cells through biolistic transformation or particle bombardment using, for instance, a gene gun. See Gao, et al., Nonviral Gene Delivery: What We Know and What Is Next, AAPS J. 2007 March; 9(1): E92-E104; Yang, et al., In vivo and in vitro gene transfer to mammalian somatic cells by particle bombardment, Proc Natl Acad Sci USA, 1990; 87:9568-9572; the contents of each of which are incorporated herein in their entirety and for all purposes. Particle bombardment through a gene gun may be used, for example, to introduce compositions of the invention into cells of the skin, mucosa, or surgically exposed tissues within a confined area. In particle bombardment methods, nucleic acid is deposited on the surface of gold particles, which are then accelerated, for example, by pressurized gas, into cells or tissue such that the momentum of the gold particles carries the nucleic acid into the cells. Id.

Particle bombardment may be performed using, for example a gene gun such as the Helios Gene Gun System available from Bio-Rad Laboratories, Inc. (Hercules, Calif.). Thus, in some embodiments, the invention provides a system for treating a viral infection that includes a gene gun 501, a vector encoding a gene for an enzyme that cuts target genetic material such as Cas9 103, and a gRNA that targets a latent virus and that has no match in the human genome 105, as shown in FIG. 6.

Magnetic nanoparticles may also be used to deliver nucleases. The basic premise is that therapeutic nucleases or their vectors are attached to, or encapsulated within, a magnetic micro- or nanoparticle. These particles may have magnetic cores with a polymer or metal coating which can be functionalized, or may consist of porous polymers that contain magnetic nanoparticles precipitated within the pores. By functionalizing the polymer or metal coating it is possible to attach, for example, therapeutic nucleic acids of the invention to target viral genome within a host cell. See Guo, et al., Recent Advances in Non-viral Vectors for Gene Delivery, Acc Chem Res. 2012 Jul. 17; 45(7): 971-979, the contents of which are incorporated herein in their entirety and for all purposes.

Compositions of the invention may be delivered by any suitable method include subcutaneously, transdermally, by hydrodynamic gene delivery, topically, or any other suitable method. In some embodiments, the composition is provided a carrier and is suitable for topical application to the human skin. The composition may be introduced into the cell in situ by delivery to tissue in a host. Introducing the composition into the host cell may include delivering the composition non-systemically to a local reservoir of the viral infection in the host, for example, topically.

Compositions of the invention may be delivered to an affected area of the skin in a acceptable topical carrier such as any acceptable formulation that can be applied to the skin surface for topical, dermal, intradermal, or transdermal delivery of a medicament. The combination of an acceptable topical carrier and the compositions described herein is termed a topical formulation of the invention. Topical formulations of the invention are prepared by mixing the composition with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed.); Ghosh, T. K.; et al. TRANSDERMAL AND TOPICAL DRUG DELIVERY SYSTEMS (1997).

The topical carriers useful for topical delivery of the compound described herein can be any carrier known in the art for topically administering pharmaceuticals, for example, but not limited to, acceptable solvents, such as a polyalcohol or water; emulsions (either oil-in-water or water-in-oil emulsions), such as creams or lotions; micro emulsions; gels; ointments; liposomes; powders; and aqueous solutions or suspensions, such as standard ophthalmic preparations.

In certain embodiments, the topical carrier used to deliver the compositions described herein is an emulsion, gel, or ointment. Emulsions, such as creams and lotions are suitable topical formulations for use in accordance with the invention. An emulsion has at least two immiscible phases, one phase dispersed in the other as droplets ranging in diameter from 0.1 μm to 100 μm. An emulsifying agent is typically included to improve stability.

In another embodiment, the topical carrier is a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995). Other suitable gels for use in the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002). Polymer thickeners (gelling agents) that may be used include those known to one skilled in the art, such as hydrophilic and hydro-alcoholic gelling agents frequently used in the cosmetic and pharmaceutical industries. Preferably the gelling agent comprises between about 0.2% to about 4% by weight of the composition. The agent may be cross-linked acrylic acid polymers that are given the general adopted name carbomer. These polymers dissolve in water and form a clear or slightly hazy gel upon neutralization with a caustic material such as sodium hydroxide, potassium hydroxide, or other amine bases.

In another preferred embodiment, the topical carrier is an ointment. Ointments are oleaginous semisolids that contain little if any water. Preferably, the ointment is hydrocarbon based, such as a wax, petrolatum, or gelled mineral oil.

In another embodiment, the topical carrier used in the topical formulations of the invention is an aqueous solution or suspension, preferably, an aqueous solution. Well-known ophthalmic solutions and suspensions are suitable topical carriers for use in the invention. The pH of the aqueous topical formulations of the invention are preferably within the range of from about 6 to about 8. To stabilize the pH, preferably, an effective amount of a buffer is included. In one embodiment, the buffering agent is present in the aqueous topical formulation in an amount of from about 0.05 to about 1 weight percent of the formulation. Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation. Preferably, the aqueous topical formulations of the invention have a viscosity in the range of from 0.015 to 0.025 Pa·s (about 15 cps to about 25 cps). The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

The topical formulations of the invention can include acceptable excipients such as protectives, adsorbents, demulcents, emollients, preservatives, antioxidants, moisturizers, buffering agents, solubilizing agents, skin-penetration agents, and surfactants. Suitable protectives and adsorbents include, but are not limited to, dusting powders, zinc sterate, collodion, dimethicone, silicones, zinc carbonate, aloe vera gel and other aloe products, vitamin E oil, allatoin, glycerin, petrolatum, and zinc oxide. Suitable demulcents include, but are not limited to, benzoin, hydroxypropyl cellulose, hydroxypropyl methylcellulose, and polyvinyl alcohol. Suitable emollients include, but are not limited to, animal and vegetable fats and oils, myristyl alcohol, alum, and aluminum acetate. Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin. Chlorine dioxide (ClO2), preferably, stabilized chlorine dioxide, is a preferred preservative for use with topical formulations of the invention. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use in the invention include, but are not limited to, acetate buffers, citrate buffers, phosphate buffers, lactic acid buffers, and borate buffers. Suitable solubilizing agents include, but are not limited to, quaternary ammonium chlorides, cyclodextrins, benzyl benzoate, lecithin, and polysorbates. Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

In certain embodiments, compounds of the invention are conjugated to nano-systems for systemic therapy, such as liposomes, albumin-based particles, PEGylated proteins, biodegradable polymer-drug composites, polymeric micelles, dendrimers, among others. See Davis et al., 2008, Nanotherapeutic particles: an emerging treatment modality for cancer, Nat Rev Drug Discov. 7(9):771-782, incorporated by reference. Long circulating macromolecular carriers, such as liposomes, can exploit the enhanced permeability and retention effect for preferential extravasation from tumor vessels. In certain embodiments, the complexes of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70. These cellular delivery systems may be introduced into the body transdermally through the methods described herein.

To deliver nucleases, hydrodynamic gene delivery may be used. This technology controls hydrodynamic pressure in capillaries to enhance endothelial and parenchymal cell permeability (Hydrodynamic Gene Delivery: Its Principles and Applications, Molecular Therapy (2007) 15 12, 2063-2069). The first clinical test of hydrodynamic gene delivery in humans was reported at the 9th Annual Meeting of the American Society of Gene Therapy (Clinical Study with Hydrodynamic Gene Delivery into Hepatocytes in Humans). Hydrodynamic gene delivery avoids potential host immune response seen in AAV delivery (Prolonged susceptibility to antibody-mediated neutralization for adeno-associated vectors targeted to the liver.).

Hydrodynamic gene delivery can also be applied to liver transplant (Hydrodynamic plasmid DNA gene therapy model in liver transplantation). Injection volumes of 40-70% of the liver weight are found to be effective in gene delivery. Combination of hydrodynamic gene delivery with targeted nuclease can potentially eliminate HBV from liver transplant, and provide more qualified organs.

The delivery of nuclease (e.g., Cas9+sgRNA) may be combined with conventional antiviral drugs, such as Lamivudine and Telbivudine. In such way, the viral load may be greatly reduced before nuclease treatment to improve treatment efficacy.

For hydrodynamic gene delivery, a composition is delivered at a pressure sufficient to generate pores in the cells proximal to the blood vessel. Hydrodynamic or energy-enhanced transdermal gene delivery are used to deliver a nucleic acid such as a plasmid that preferably encodes an nuclease enzyme. In a preferred embodiment, the enzyme is Cas9.

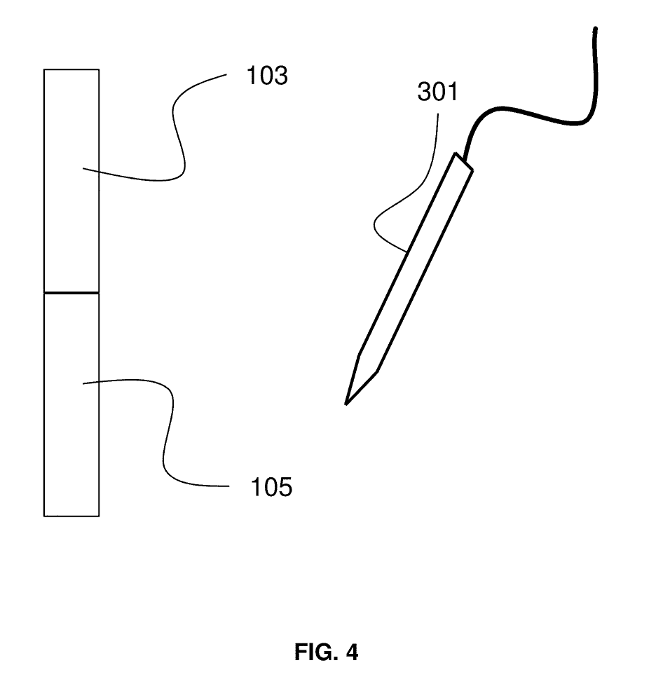

FIG. 4 diagrams a plasmid according to certain embodiments.

Where the viral genome is a hepatitis B genome, the plasmid may contain genes for one or more sgRNAs targeting locations in the hepatitis B genome such as PreS1, DR1, DR2, a reverse transcriptase (RT) domain of polymerase, an Hbx, and the core ORF. In a preferred embodiment, the one or more sgRNAs comprise one selected from the group consisting of sgHBV-Core and sgHBV-PreS1.

For hydrodynamic gene delivery, the composition may be delivered via an intravascular delivery catheter, e.g., by navigating a balloon catheter to the blood vessel at a target location in the subject, inflating the balloon, and delivering the composition via a lumen in the balloon catheter.

In certain embodiments, the Cas9 and gRNA complex may be delivered to cells as a nucleic acid (e.g., plasmid or mRNA). Commercially available kits for mRNA transfection are available, for example, from Mirus Bio LLC (Madison, Wis.) and ThermoFisher Scientific Inc. (Waltham, Mass.). Delivery as an RNP affords good control over dosing and may be desirable to reduce immunogenicity through careful control of exposure to foreign compounds while mRNA provides better transfection and more effective treatment as the protein is continuously synthesized. In various embodiments, the delivery format may be chosen based on the cell type being delivered to and the disease being treated.

In some embodiments, the invention provides a composition for topical application (e.g., in vivo, directly to skin of a person). The composition may be applied superficially (e.g., topically). The composition provides a nuclease or gene therefore and includes a pharmaceutically acceptable diluent, adjuvant, or carrier. Preferably, a carrier used in accordance with the subject invention is approved for animal or human use by a competent governmental agency, such as the US Food and Drug Administration (FDA) or the like. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. These formulations contain from about 0.01% to about 100%, preferably from about 0.01% to about 90% of the MFB extract, the balance (from about 0% to about 99.99%, preferably from about 10% to about 99.99% of an acceptable carrier or other excipients. A more preferred formulation contains up to about 10% MFB extract and about 90% or more of the carrier or excipient, whereas a typical and most preferred composition contains about 5% MFB extract and about 95% of the carrier or other excipients. Formulations are described in a number of sources that are well known and readily available to those skilled in the art.

In certain embodiments, HBV may be used as a delivery vehicle for Cas9 genes. See Deng et al., 2009, Hepatitis B virus as a gene delivery vector activating foreign antigenic T cell response that abrogates viral expression in mouse models, Hepatology 50(5):1380, incorporated by reference. The HBV core used for delivery may also be modified to reduce the HBV specific immunogenicity of this delivery method.

### Ex Vivo Delivery

In certain embodiments, compounds of the invention may be delivered in vitro to extracted cells or tissues before transplantation back into the donor or another recipient. By administering treatment directly to the removed cells and tissue, global toxicity and immunogenicity may be avoided and treatment may be better tailored and delivered to the target tissue. Examples of methods for applying ex-vivo treatments are discussed below.

Compositions and methods of the invention may be applied, in vitro to mixed populations of cells and tissues including whole organs.

Methods of the invention may include obtaining a cell from a donor and delivering to the cell a nuclease that cleaves viral nucleic acid. The cell is then provided for transplantation to a patient.

In various examples, in vitro treatment, followed by implantation may be performed on a patient's blood, B cells, or stem cells. It should be appreciated that any type of cell may be obtained from a donor. For example, exocrine secretory epithelial cells, hormone secreting cells, epithelial cells, sensory transducer cells, neuron cells, glial cells, lens cells, hepatocyte cells, adipocyte cells, lipocyte cells, kidney cells, liver cells, prostate gland cells, pancreatic cells, ameloblast epithelial cells, planum semilunatum epithelial cells, organ of Corti interdental epithelial cells, loose connective tissue fibroblasts, corneal fibroblasts (corneal keratocytes), tendon fibroblasts, bone marrow reticular tissue fibroblasts, pericytes, nucleus pulposus cells, odontoblast/odontocytes, chondrocytes, osteoprogenitor cells, hyalocytes, stellate cells, hepatic stellate cells, skeletal muscle cells, satellite cells, heart muscle cells, smooth muscle cells, myoepithelial cells, myoepithelial cells, erythrocytes, megakaryocytes, monocytes, connective tissue macrophages, epidermal Langerhans cells, osteoclasts, dendritic cells, microglial cells neutrophil granulocytes, eosinophil granulocytes, basophil granulocytes, hybridoma cells, mast cells, helper T cells, suppressor T cells, cytotoxic T cells, natural killer T cells, B cells, natural killer cells reticulocytes, somatic stem cells, embryonic stem cells, or hematopoietic stem cells may be used in methods of the invention. In some embodiments, the cell is infected with a virus and contains viral nucleic acid within the cell. The virus may be a herpes family virus. In some embodiments, the virus is in the latent stage in the cell.

Cells for use in the methods of the invention may be obtained from any suitable source. In a preferred embodiment, cells are obtained from a donor, who may be chosen based on being a suitable donor for a patient who will need a bone marrow transplant or other infusion of HSCs. Preferably, the donor is a known family member of the patient, and may even be the patient him- or her-self. For example, a patient may provide their own cells for later delivery in a transplant procedure. E.g., cells may be obtained from an umbilical cord sample taken from the patient and stored, and then treated according to methods of the invention prior to transplant/implantation into the patient.

Any type of cell may be used in the methods of the invention. Cells may be eukaryote, prokaryote, mammalian, human, etc. In some embodiments, stem cells are used in the methods of the invention. Stem cells may be obtained from a stem cell bank, which are ultimately derived from a donor, or directly from a donor. Stem cells may be harvested, purified, and treated by any known method in the art.

Stem cells may be harvested from a donor by any known methods in the art. For example, U.S. Pub. 2013/0149286 details procedures for obtaining and purifying stem cells from mammalian cadavers. Stem cells may be harvested from a human by bone marrow harvest or peripheral blood stem cell harvest, both of which are well known techniques in the art. After stem cells have been obtained from the source, such as from certain tissues of the donor, they may be cultured using stem cell expansion techniques. Stem cell expansion techniques are disclosed in U.S. Pat. No. 6,326,198 to Emerson et al., entitled “Methods and compositions for the ex vivo replication of stem cells, for the optimization of hematopoietic progenitor cell cultures, and for increasing the metabolism, GM-CSF secretion and/or IL-6 secretion of human stromal cells,” issued Dec. 4, 2001; U.S. Pat. No. 6,338,942 to Kraus et al., entitled “Selective expansion of target cell populations,” issued Jan. 15, 2002; and U.S. Pat. No. 6,335,195 to Rodgers et al., entitled “Method for promoting hematopoietic and mesenchymal cell proliferation and differentiation,” issued Jan. 1, 2002, which are hereby incorporated by reference in their entireties. In some embodiments, stem cells obtained from the donor are cultured in order to expand the population of stem cells. In other preferred embodiments, stem cells collected from donor sources are not expanded using such techniques. Standard methods can be used to cyropreserve the stem cells.

In embodiments of the invention, either embryonic or adult stem cells may be used. Adult stem cells, also known as somatic stem cells, may be found in organs and tissues of the donor. For example, the central nervous system, bone marrow, peripheral blood, blood vessels, umbilical cordon blood, skeletal muscle, epidermis of the skin, dental pulp, heart, gut, liver, pancreas, lung, adipose tissue, ovarian epithelium, retina, cornea and testis. Somatic stem cells include, but are not limited to, mesenchymal stem cells, hematopoietic stem cells, skin stem cells, and adipose-derived stromal stem cells. The stem cells may be undifferentiated, or they may be differentiated.

Methods of the invention include providing the cell for transplant into the patient. In some embodiments, the treated cells are labeled, stored, shipped, or otherwise readied for medical use. In certain embodiments, methods of the invention include delivering the cell or cells into the body of the patient.

In some embodiments, hematopoietic stem cell transplantation (HSCT) involves the intravenous (IV) infusion of autologous or allogeneic stem cells to reestablish hematopoietic function in patients whose bone marrow or immune system is damaged or defective. Hematopoietic stem cell transplantation (HSCT) requires the extraction (apheresis) of haematopoietic stem cells (HSC) from the patient and storage of the harvested cells in a freezer. The patient is then treated with high-dose chemotherapy with or without radiotherapy with the intention of eradicating the patient's malignant cell population at the cost of partial or complete bone marrow ablation (destruction of patient's bone marrow function to grow new blood cells). The patient's own stored stem cells are then treated with nucleases according to methods of the invention, and then transfused into his/her bloodstream, where they replace destroyed tissue and resume the patient's normal blood cell production.

In some embodiments, allogeneic HSCT, which involves a healthy donor and the patient recipient, incorporate methods of the invention. Allogeneic HSC donors must have a tissue (HLA) type that matches the recipient. Matching is performed on the basis of variability at three or more loci of the HLA gene, and a perfect match at these loci is preferred. Allogeneic transplant donors may be related (usually a closely HLA matched sibling), syngeneic (a monozygotic or ‘identical’ twin of the patient—necessarily extremely rare since few patients have an identical twin, but offering a source of perfectly HLA matched stem cells) or unrelated (donor who is not related and found to have very close degree of HLA matching). Unrelated donors may be found through a registry of bone marrow donors such as the National Marrow Donor Program. In general, by transfusing healthy stem cells to the recipient's bloodstream to reform a healthy immune system, allogeneic HSCTs may improve chances for cure or long-term remission once the immediate transplant-related complications are resolved.

Cells harvested or obtained may be frozen (cryopreserved) for prolonged periods without damaging the cells. In some embodiments, the cells may be harvested from the recipient or donor months or years in advance of the transplant treatment. To cryopreserve HSC, a preservative, DMSO, may be added, and the cells may be cooled very slowly in a controlled-rate freezer to prevent osmotic cellular injury during ice crystal formation. HSC may be stored for years in a cryofreezer, which typically uses liquid nitrogen.

Providing for medical use can include labeling, storing, shipping, or otherwise readying for use. In a preferred embodiment, providing the cells for transplant into the patient includes putting the cells in a container, such as the blood collection tube sold under the trademark VACUTAINER by BD (Franklin Lakes, N.J.) that is labeled with information that can be used to identify the recipient. The container may be stored for a period of time until the cells are needed for transplantation. In some embodiments, providing the cells for transplant into the patient includes holding the cells in a container after delivering a nuclease.

Delivering into the patient may include delivering viral-free cells into a patient by intravenous (IV) infusion. In other embodiments, the viral-free cells may be transplanted into a patient via a surgery, or by placing the sample into a location in the patient's body. In other embodiments, the cells are placed into a patient during a surgical procedure.

In some embodiments, tissues, such as organs are treated with a nuclease complex prior to transplantation. Cells and tissues treated with a nuclease according to the methods of the invention may have been removed from a patient before treatment and may then be provided for transplantation after treatment. Tissues or organs may be transplanted into the original donor after treatment such as in cases where treatment is more easily accomplished ex-vivo. Alternatively, organs or tissues from donors may be treated prior to transplantation into a separate recipient. In some embodiments, organs are treated with the nuclease to render the tissue free of one or more viral infections, prior to transplantation.

In some embodiments of the invention, the nucleases are prepared for use in organs for transplant. Organ transplantation is the moving of an organ from one body to another or from a donor site to another location on the person's own body, to replace the recipient's damaged or absent organ. Organ can also be created or re-grown from the person's own cells (stem cells, or cells extracted from the failing organs) or from cells of another person. Organs can either be from a living or cadaveric source. Organs that can be transplanted are the heart, kidneys, liver, lungs, pancreas, intestine, and thymus. Tissues include bones, tendons (both referred to as musculoskeletal grafts), cornea, skin, heart valves, nerves and veins. Cornea and musculoskeletal grafts are the most commonly transplanted tissues, or organs.

In various embodiments, delivery of the Cas9-type/guide RNA complex may be to a variety of tissues as noted above. Treatment may be varied according to the disease to be treated and the location of the cells to be treated. Delivery can be to any tissue in vivo, including to tissue surfaces, intra-tumor surfaces, and organ surfaces. Delivery can be via any route, such as inter arterial for (e.g., for pulmonary tissues), intravenous (e.g., for liver tissue), or transmucosally.

In certain embodiments compositions and methods may be used to treat oncoviruses and cancers resulting therefrom such as nasopharyngeal carcinoma (NPC). In NPC, B cells may migrate close to epithelial surface which allows for direct application to the effected tissue. Other diseases may require targeting of circulating B cells, which are harder to access and treat. In certain embodiments, B cells may be removed and treated in vitro as described above before being delivered back into the patient. Other virus related cancers treatable with methods and compounds of the invention include gastrointestinal carcinoma and lethal midline granuloma.

In certain embodiments, delivery methods may be adapted to the cell type at which a nuclease is being provided.

### Targeted Treatment of Viral Infections Using Nuclease

According to certain methods of the invention, nucleases may be used to target a viral genome in an infected cell with reduced immunogenicity and toxicity to the infected cell. Once inside the cell, the nuclease cuts the viral genome. In addition to latent infections this invention can also be used to control actively replicating viruses by targeting the viral genome before it is packaged or after it is ejected. In some embodiments, methods and compositions of the invention use a nuclease such as Cas9 to target latent viral genomes, thereby reducing the chances of proliferation.

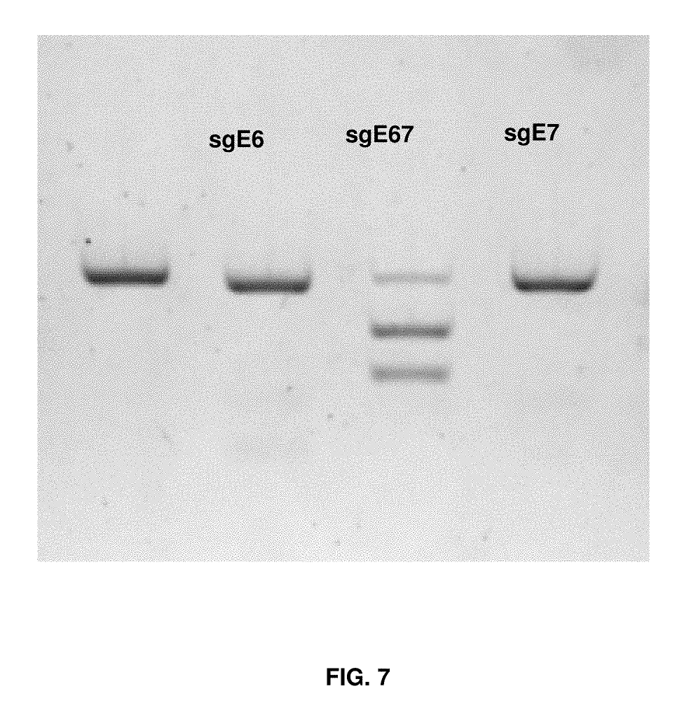

FIG. 7 shows the results of successfully cleaving HPV genome using Cas9 nuclease, a gRNA for E6, and a gRNA for E7. The nuclease forms a complex with the gRNA (e.g., crRNA+tracrRNA or sgRNA). The complex cuts the viral nucleic acid in a targeted fashion to incapacitate the viral genome. The Cas9 nuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even if natural repair mechanisms join the genome together, the genome is render incapacitated.

The nuclease, or a gene encoding the nuclease, may be delivered into an infected cell by any of the methods discussed above. For example, the infected cell can be transfected with DNA that encodes Cas9 and gRNA (on a single piece or separate pieces). The gRNAs are designed to localize the Cas9 nuclease at one or several locations along the viral genome. The Cas9 nuclease causes double strand breaks in the genome, causing small fragments to be deleted from the viral genome. Even with repair mechanisms, the deletions render the viral genome incapacitated.

It will be appreciated that method and compositions of the invention can be used to target viral nucleic acid without interfering with host genetic material. Methods and compositions of the invention employ a targeting moiety such as a guide RNA that has a sequence that hybridizes to a target within the viral sequence. Methods and compositions of the invention may further use a targeted nuclease such as the cas9 enzyme, or a vector encoding such a nuclease, which uses the gRNA to bind exclusively to the viral genome and make double stranded cuts, thereby removing the viral sequence from the host.

Where the targeting moiety includes a guide RNA, the sequence for the gRNA, or the guide sequence, can be determined by examination of the viral sequence to find regions of about 20 nucleotides that are adjacent to a protospacer adjacent motif (PAM) and that do not also appear in the host genome adjacent to the protospacer motif.

Preferably a guide sequence that satisfies certain similarity criteria (e.g., at least 60% identical with identity biased toward regions closer to the PAM) so that a gRNA/cas9 complex made according to the guide sequence will bind to and digest specified features or targets in the viral sequence without interfering with the host genome. Preferably, the guide RNA corresponds to a nucleotide string next to a protospacer adjacent motif (PAM) (e.g., NGG, where N is any nucleotide) in the viral sequence. Preferably, the host genome lacks any region that (1) matches the nucleotide string according to a predetermined similarity criteria and (2) is also adjacent to the PAM. The predetermined similarity criteria may include, for example, a requirement of at least 12 matching nucleotides within 20 nucleotides 5′ to the PAM and may also include a requirement of at least 7 matching nucleotides within 10 nucleotides 5′ to the PAM. An annotated viral genome (e.g., from GenBank) may be used to identify features of the viral sequence and finding the nucleotide string next to a protospacer adjacent motif (PAM) in the viral sequence within a selected feature (e.g., a viral replication origin, a terminal repeat, a replication factor binding site, a promoter, a coding sequence, or a repetitive region) of the viral sequence. The viral sequence and the annotations may be obtained from a genome database.

Where multiple candidate gRNA targets are found in the viral genome, selection of the sequence to be the template for the guide RNA may favor the candidate target closest to, or at the 5′ most end of, a targeted feature as the guide sequence. The selection may preferentially favor sequences with neutral (e.g., 40% to 60%) GC content. Additional background regarding the RNA-directed targeting by nuclease is discussed in U.S. Pub. 2015/0050699; U.S. Pub. 20140356958; U.S. Pub. 2014/0349400; U.S. Pub. 2014/0342457; U.S. Pub. 2014/0295556; and U.S. Pub. 2014/0273037, the contents of each of which are incorporated by reference for all purposes. Due to the existence of human genomes background in the infected cells, a set of steps are provided to ensure high efficiency against the viral genome and low off-target effect on the human genome. Those steps may include (1) target selection within viral genome, (2) avoiding PAM+target sequence in host genome, (3) methodologically selecting viral target that is conserved across strains, (4) selecting target with appropriate GC content, (5) control of nuclease expression in cells, (6) vector design, (7) validation assay, others and various combinations thereof. A targeting moiety (such as a guide RNA) preferably binds to targets within certain categories such as (i) latency related targets, (ii) infection and symptom related targets, and (iii) structure related targets.

Host cells may grow at different rate, based on the specific cell type and expression may be adjusted accordingly. High nuclease expression is necessary for fast replicating cells, whereas low expression may help in avoiding off-target cutting in non-infected cells. If the nuclease is expressed from a vector, having the viral replication origin in the vector can increase the vector copy number dramatically, only in the infected cells. Each promoter has different activities in different tissues. Gene transcription can be tuned by choosing different promoters. Transcript and protein stability can also be tuned by incorporating stabilizing or destabilizing (ubiquitin targeting sequence, etc) motif into the sequence.

Specific promoters may be used for the gRNA sequence, the nuclease (e.g., cas9), other elements, or combinations thereof. For example, in some embodiments, the gRNA is driven by a U6 promoter. A vector may be designed that includes a promoter for protein expression (e.g., using a promoter as described in the vector sold under the trademark PMAXCLONING by Lonza Group Ltd (Basel, Switzerland). A vector may be a plasmid (e.g., created by synthesis instrument 255 and recombinant DNA lab equipment). In certain embodiments, the plasmid includes a U6 promoter driven gRNA or chimeric guide RNA (sgRNA) and a ubiquitous promoter-driven cas9. Optionally, the vector may include a marker such as EGFP fused after the cas9 protein to allow for later selection of cas9+ cells. It is recognized that cas9 can use a gRNA (similar to the CRISPR RNA (crRNA) of the original bacterial system) with a complementary trans-activating crRNA (tracrRNA) to target viral sequences complementary to the gRNA. It has also been shown that cas9 can be programmed with a single RNA molecule, a chimera of the gRNA and tracrRNA. The single guide RNA (sgRNA) can be encoded in a plasmid and transcription of the sgRNA can provide the programming of cas9 and the function of the tracrRNA. See Jinek, 2012, A programmable dual-RNA-guided DNA nuclease in adaptive bacterial immunity, Science 337:816-821 and especially FIG. 5A therein for background, incorporated by reference.

Examples of various viruses, the nucleic acid of which is to be targeted by the targeting polypeptide, include but are not limited to, herpes simplex virus (HSV)-1, HSV-2, varicella zoster virus (VZV), Epstein-Barr virus (EBV), cytomegalovirus (CMV), human herpesvirus (HHV)-6A and -6B, HHV-7, Kaposi's sarcoma-associated herpesvirus (KSHV), human polyomavirus, Merkel cell polyomavirus (MCV), JC virus, BK virus, parvovirus b19, adeno-associated virus (AAV), adenovirus, Human papillomavirus (HPV), JC virus, Smallpox, Hepatitis B virus, Human bocavirus, Human astrovirus, Norwalk virus, coxsackievirus, hepatitis A virus, poliovirus, rhinovirus, severe acute respiratory syndrome virus, Hepatitis C virus, yellow fever virus, dengue virus, West Nile virus, Rubella virus, Hepatitis E virus, Human immunodeficiency virus (HIV), Influenza virus, Guanarito virus, Junin virus, Lassa virus, Machupo virus, Sabiá virus, Crimean-Congo hemorrhagic fever virus, Ebola virus, Marburg virus, Measles virus, Mumps virus, Parainfluenza virus, Respiratory syncytial virus (RSV), Human metapneumovirus, Hendra virus, Nipah virus, Rabies virus, Hepatitis D, Rotavirus, Orbivirus, Coltivirus, and Banna virus. In one embodiment, the virus is a member of the herpesviridae family, e.g., herpes simplex virus (HSV)-1, HSV-2, varicella zoster virus (VZV), Epstein-Barr virus (EBV), cytomegalovirus (CMV), human herpesvirus (HHV)-6A and -6B, HHV-7, and Kaposi's sarcoma-associated herpesvirus (KSHV).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for delivering a nuclease to cells, the method comprising:
providing to cells a first nuclease that cuts a target site in a target nucleic acid; and
providing to the cells a second nuclease that cuts the target site, wherein the first nuclease and the second nuclease do not generate the same antigenic response.

2. The method of claim 1, wherein the target nucleic acid is viral nucleic acid from a virus infecting the cells.

3. The method of claim 2, wherein the cells are in a patient being treated for the virus infecting the cells.

4. The method of claim 3, wherein the method further comprises administering an immunosuppressant to the patient.

5. The method of claim 2, wherein the first nuclease and the second nuclease each have at least 80% sequence identity to Cas9 and wherein the first nuclease and the second nuclease do not have 100% sequence identity to each other.

6. The method of claim 2, wherein the first nuclease and the second nuclease are different nucleases selected from the group consisting of: Cas9, Cas6, Cpf1, and modified versions thereof.

7. The method of claim 6, wherein the first or second nuclease comprises a modified nuclease not known to occur in nature.

8. The method of claim 7, wherein the modified nuclease is smaller than a wild type counterpart.

9. The method of claim 8, wherein the modified nuclease has been modified by removal of nonfunctional structures of the wild type counterpart.

10. The method of claim 7, wherein the modified nuclease has an altered charge or hydrophobicity from a wild type counterpart.

11. The method of claim 7, wherein the modified nuclease is a fusion protein comprising a portion of a protein selected from the group consisting of: GFP, Fc, and IgG.

12. The method of claim 1, wherein the first nuclease and the second nuclease originate from different species.

13. The method of claim 1, wherein the first nuclease and second nuclease are provided by delivering nucleic acids that encode the first nuclease and the second nuclease.

14. The method of claim 13, wherein nucleic acids are each DNA vectors that each encode a guide RNA complementary to the nucleic acid target, wherein the first nuclease and the second nuclease each form a complex a transcript of the guide RNA to specifically cut the target site.

15. The method of claim 1, wherein the cells comprise a mixture of cell types.

16. The method of claim 1, further comprising:
assaying for viral load in the cells and determining an amount of the first or second nuclease to deliver based on the viral load.

17. The method of claim 1, wherein first nuclease and the second nuclease are each delivered encoded by a nucleic acid that is introduced into the cell using one selected from the group consisting of: lipid nanoparticle, and a liposome.
